(19) United States
(12) Patent Application Publication
(10) Pub. No.: US 2026/0199050 A1
(43) Pub. Date: Jul. 16, 2026

(54) ACCESS PORT ASSEMBLY, FLOW CONTROL SYSTEM AND TRAJECTORY DETERMINATION ASSEMBLY, AND METHODS FOR USING THE AFOREMENTIONED

(71) Applicants: B. G. NEGEV TECHNOLOGIES AND APPLICATIONS LTD, Beer Sheva (IL); CHILDREN'S HOSPITAL MEDICAL CENTER

(72) Inventors: Francesco MANGANO, Cincinnati, OH (US); David ZARROUK, BEER SHEVA (IL); Atai DANIEL, BEER SHEVA (IL); Segev PEER, BEER SHEVA (IL); Ben GRINSHPAN, BEER SHEVA (IL); Matan CORONEL, BEER SHEVA (IL)

(21) Appl. No.: 19/135,867

(22) PCT Filed: Dec. 6, 2023
(86) PCT No.: PCT/IB2023/062295

§ 371 Date: Jun. 5, 2025

Related U.S. Application Data
(60) Provisional application No. 63/430,495, filed on Dec. 6, 2022.

(51) Int. Cl. A61B 90/13; A61B 17/16; A61B 90/10

(52) U.S. Cl. CPC A61B 90/13; A61B 17/1662; A61B 2090/103

(57) ABSTRACT

Embodiments pertain to an access port assembly configured to assist in creating access for and accommodating a medical device extending from outside a patient body and terminating in a cavity internal to the patient body. The assembly may comprise a guide element comprising a guide channel having a longitudinal guide axis extending from an inlet to an outlet of the guide element for guiding the medical device through the guide channel. The guide element may be movably coupled with the fixation base by an articulating linkage to allow selecting an orientation of the guide channel relative to the site of interest of the patient body. The articulating linkage may be implemented as a ball-and-socket linkage. Embodiments also pertain to a system for controlling delivery of fluid into the patient body and/or removal of fluid from the patient body, via one or more access ports.

### CROSS-REFERENCE TO RELATED APPLICATIONS

This application claims priority and/or benefit from U.S. patent application 63/430,495 filed Dec. 6, 2022, titled “ACCESS PORT ASSEMBLY, FLOW CONTROL SYSTEM AND TRAJECTORY DETERMINATION ASSEMBLY, AND METHODS FOR USING THE AFOREMENTIONED”, which is incorporated herein by reference in its entirety.

### BACKGROUND

The implantation of an external ventricular drain is perhaps the most common procedure performed by both pediatric and adult neurosurgeons throughout the world. The procedure is done in different healthcare delivery locations such as the emergency room, the operating room, and even at the bedside at times. Some indications are emergent and lifesaving while others are done in more controlled environments for diversion of cerebrospinal fluid.

The description above is presented as a general overview of related art in this field and should not be construed as an admission that any of the information it contains constitutes prior art against the present patent application.

### BRIEF DESCRIPTION OF THE DRAWINGS

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The number of elements shown in the Figures should by no means be construed as limiting and is for illustrative purposes only. The figures are listed below.

FIG. 1A is a schematic view of an access port assembly including an articulated linkage, according to some embodiments.

FIG. 1B is a schematic view of the access port assembly operably engaged with a cranium of a patient, according to some embodiments.

FIG. 1C is a schematic side view of the access port assembly with the articulated linkage in a tilted orientation, according to some embodiments.

FIG. 2 is a schematic front view of the access port assembly, according to some embodiments.

FIG. 3 is a schematic top view of the access port assembly, according to some embodiments.

FIG. 4 is a schematic exploded view of the access port assembly, according to some embodiments.

FIGS. 5A and 5B schematically illustrate an open and a locked state, respectively, of an articulating linkage of the access port assembly, according to some embodiments.

FIGS. 6A and 6B schematically illustrate top views of an access port assembly in a locked and unlocked state, according to some embodiments.

FIG. 7 schematically illustrates another view of the secured state of the articulating linkage of the access port assembly employed in conjunction with a medical device operably engaged with the access port assembly, according to some embodiments.

FIGS. 8A to 8C schematically illustrate a top, cross sectional and isometric view, respectively, of a ball of a ball-and-socket implemented articulating linkage, according to some embodiments.

FIG. 9 schematically illustrates a guide element in operable engagement with the articulating linkage of the access port assembly, according to some embodiments.

FIGS. 10A and 10B schematically illustrate a top and isometric view, respectively, of the guide element of the access port assembly, according to some embodiments.

FIGS. 11A and 11B schematically illustrate cross-sectional view of the guide element in operable deployment without and with a guide locking member, respectively, according to some embodiments.

FIG. 11C schematically illustrates a guide element in alignment relative to a ball member of the articulating linkage, according to some alternative embodiments.

FIGS. 12A and 12B schematically illustrate a medical device deployed through the guide element with the guide locking member is in an unsecured and secured state, respectively, according to some embodiments.

FIG. 13 schematically illustrate the insertion and fixation of medical devices through the guides in a plurality of access port assemblies, according to some embodiments.

FIG. 14 schematically illustrates a flow control system, according to some embodiments.

FIG. 15 schematically illustrates a trajectory determination assembly, according to some embodiments.

FIGS. 16A and 16B schematically illustrate the use of the trajectory determination assembly with top and isometric view, respectively, where the trajectory determination assembly is coupled with an access port apparatus, according to some embodiments.

FIG. 16C schematically illustrates a trajectory determination assembly, according to some alternative embodiments.

### DETAILED DESCRIPTION

The current standard of care of external ventricular drain (EVD) is to place the catheter in a relatively blind fashion based on standard craniofacial morphometric measurements to identify an entry site (e.g., Kocher's Point) and a typical trajectory to enter the ventricle. The procedure may be relatively straightforward when the ventricles are extremely large. However, in many cases the trajectory of the catheter is not as accurate when the ventricles are not enlarged, even in cases where the ventricles are enlarged. Therefore, the catheter may still mistakenly be deployed to a less than ideal position within the ventricular system and at times could miss the ventricles altogether. This may occur, in particular, if the procedure is performed by interns and less experienced surgeons. As a result of suboptimal or faulty catheter deployment, EVD malfunctions (or even neurologic injury) could occur, which the great majority of the time can only be rectified with additional surgeries. In addition, further difficulties are encountered when the primary diagnosis is intraventricular hemorrhage. In these cases, the EVD is expected to washout blood clots and bloody CSF which can block flow through the catheter. Once flow is inhibited the EVD typically needs to be manipulated and removed/replaced to re-establish flow and normalize intracranial pressure.

It is noted that embodiments discussed herein may be employed in conjunction with additional or alternative entry point identification procedures for identifying an (e.g., cranial) entry point. Such entry point identification procedures may be based or include the employment of one or more imaging modalities such as, for example, CT and/or MRI, in addition or as an alternative to standard craniofacial morphometric measurements.

Aspects of embodiments pertain to an access port assembly for providing access to a site of interest internal to a patient body, e.g., of humans or other mammals, and to a (e.g., intracranial) flow and/or pressure control system for circulating fluid via a plurality of access port assemblies. In addition, aspects of embodiments pertain to methods for employing and/or using the access port assembly for operably engaging one or more access assemblies with a site of interest internal to the patient body.

In addition, embodiments pertain a trajectory determination assembly configured to assist in determining a desired trajectory of, e.g., a cranial catheter and/or any other suitable medical device, relative to the patient body for operable deployment or engagement with the access port assembly, to facilitate and ensure desired clinical engagement of the medical device with a site of interest internal to the patient body. For example, the trajectory determination assembly assists in determining a desired trajectory of a ventricular drainage catheter through a cranial target site, e.g., for use in conjunction with the access port assembly.

In some embodiments, the access port assembly and/or the flow control system, once operably coupled with the patient body, may be employed for performing one or more medical procedures including, for example, brain tissue biopsy, aspiration of blood clot, instilling medication in a specific lesion or compartment of the brain, and/or washout of (e.g., infected) fluid.

Embodiments of the present access port assembly are configured to assist in creating access for, and the accommodation of, one or more medical devices (e.g., catheter) extending from outside a patient body and terminating inside to the patient body, e.g., within or near a patient site of interest such as in or near a brain region.

More particularly, the present access port assembly relates to fixation of a guide for the medical device with respect to an identified desired entry point and desired trajectory. In addition, embodiments pertain to a control system for controlling (e.g., modulating) fluid input flow and/or fluid output flow via at least two access port assemblies.

Although embodiments are discussed herein with respect to providing cranial access, this should by no means be construed in a limiting manner. Hence, the access port assembly may be employed for creating access to a region internal to the patient body that is different from an intracranial body region. For example, the access port assembly may be employed for facilitating and/or creating access into an abdominal cavity. Further applications may include the employment of the assembly in conjunction with, for example, endoscopic third ventriculostomy procedures. In such procedure the assembly can be used for guiding an endoscope into the ventricle. That trajectory can then be locked, and the procedure can be done to make a hole at the bottom of the third ventricle through the inserted endoscope.

In some embodiments, the access port assembly may be configured to be fixedly coupled external to the patient body and allow for setting a medical device operably coupled to the access port into a desired trajectory and, optionally, at a desired distance relative to the patient's site of interest.

The access port assembly may comprise a fixation base (also: base plate) with at least one base fastener for securely coupling the access port assembly at a selected position to a site of interest external to the patient body.

The access port assembly may further include an articulating linkage (e.g., ball-and-socket articulating linkage) comprising a first articulating member (e.g., the socket) that is operably coupled with a second articulating member (e.g., the ball). The second articulating element may constitute or include a guide channel having a longitudinal guide element axis.

While the embodiments discussed herein refer to an articulating element with a sphere shape geometry including an articulating channel or guide channel having a longitudinal guide axis, this should by no means be construed as limiting. For example, in some examples, the “ball-member” of a ball-and-socket implemented articulating linkage may have a substantially spherical shape or a non-spherical shape such as, for example, the shape of a polyhedrons (e.g., octahedron, dodecahedron, icosahedron etc.). In some examples, the articulating channel may have a variety of cross-sectional (e.g., non-circular) geometric shapes, such as a hexagon, pentagon, rectangle, or a triangular cross-sectional channel shapes.

The orientations of the articulating members relative to each other may be determined in accordance with clinical operational needs.

The guide channel may extend from an inlet to an outlet of the articulating element (or of the guide element, for that matter) for guiding the medical device through the guide channel, e.g., once the guide channel is set in a desired orientation.

The access port assembly further includes an orientation locking mechanism for securing the articulating first member (or the guide element) in a selected orientation relative to patient site of interest (that may be internal and/or external to the patient body), or any other reference frame, which can be associated with to a part of the patient's body such as, for example, the patient's head, abdomen. In some examples, the reference may be associated with a patient-carrying platform, and/or the like, for example for use in conjunction with stereotactic procedures.

In some embodiments, the selected orientation may define a trajectory for inserting a medical device into the patient's body.

In some examples, a single locking mechanism (e.g., a single handle) may be employed for securing the second articulated member in a desired orientation relative to the first articulated member.

In some embodiments, a locking mechanism for securing a guide channel in a desired orientation may include a locking handle, which upon lowering the handle towards the fixation base, causes a pin to engage with the guide element, thereby exerting pressure on the accommodating articulating element or guide element, inhibiting movement of the guide element from the desired orientation relative to the reference frame.

In some embodiments, a locking mechanism comprises a locking handle, which upon lowering the handle towards the fixation base, exerts pressure on the articulating element and inhibit movement from desired orientation relative to the reference frame.

In some embodiments, instead of or in addition to a threaded mechanism, the locking mechanism may include or be based on other mechanism for securing (e.g., form fittingly and/or frictionally locking) the guide element in a desired orientation including, for example, friction latches, snap fasteners, bayonet mount, slide-on/push-on coupling, and/or the like.

In some embodiments, instead of or in addition to a threaded mechanism, the locking mechanism may include or be based on other mechanism for securing (e.g., form fittingly and/or frictionally locking) the guide element in a desired orientation including, for example, friction latches, snap fasteners, bayonet mount, slide-on/push-on coupling, and/or the like.

In some examples, a slide-locking (e.g., locking cap) mechanism may be employed in conjunction with the second articulated member to secure the medical device (e.g., a catheter, an endoscope, etc.) in position along the articulating guide axis, e.g., as described herein below in more detail.

In some embodiments, at least part of a guide channel's diameter may be adaptable to enable the deployment and housing of devices having diameters of different magnitudes (e.g., endoscopes, catheters). In some embodiments, a kit may be provided that includes assembly elements which are interchangeably selectable according to the device's diameter to be deployed.

In some embodiments, the orientation locking mechanism may also function, concurrently, as a slide-locking mechanism. For example, a same single locking mechanism may be employed for securing or locking the guide channel in a desired orientation relative to a reference frame and for locking the medical device in a desired position (e.g., distance) relative to a patient site of interest external and/or internal to a patient body. For example, a locking mechanism may secure the second articulating element (e.g., the ball), which may include a guide channel or to which the guide channel may be fixedly coupled, in a desired orientation for guiding a device in a desired trajectory towards a patient site of interest. In addition, the same or a different locking mechanism may be configured for securing the medical device at a desired position with respect to a position along the longitudinal guide axis of the guide element. In some embodiments, the guide channel may be implemented as a bore in the second articulating element (e.g., the ball). In some examples, the guide member may be a separate element that is selectively insertable into and removable from the second articulating element and which may include an exchangeable sleeve shaped base, for example, as outlined herein in more detail. The holder member may be operably deployable with respect to the second articulating element, e.g., by inserting the holder member into the channel formed in the second articulating element. For example, the access port assembly may be configured such that at least one additional guide element can be operably mounted on the second articulating element, and the medical device may be operably secured with respect to the additional guide element in a desired orientation relative to a reference frame and at a desired distance from the patient site of interest to assure a clinically desired engagement of the medical device relative the patient body.

The additional guide element and/or the medical device can be mounted and secured in place with respect to the second articulating element (e.g., ball) member, by a variety of fitting connectors including, for example, various hose/tube connectors, etc.

In some embodiments, to finalize the placement of the medical device through the guide element and into the patient body, a locking cap is introduced.

In one example implementation, the second articulating element may be configured for, e.g., directly frictionally and/or otherwise securing the medical device in a desired position relative to the bore's (e.g., the guide channel's) guide axis, by suitable engagement of the locking mechanism. For example, the guide element and/or the medical device may be configured to comprise an elastic interference fit closure mechanism, e.g., press-fit connector.

In another embodiment, a guide holder member (also: additional guide element) may be configured for allowing frictionally securing the medical device in a desired position relative to the guide channel's guide axis. For example, the additional guide element and/or the second articulating member may be configured for (e.g., frictionally and/or form-fittingly) coupling the additional guide element with the second articulating element, and the additional guide element and/or the medical device may be configured such that the medical device can be secured in place in the additional guide element.

In some embodiments, the guide element (whether implemented as part of the second articulating element or as an additional element) may be configured to include various connector mechanisms including, for example, an elastic interference fit closure mechanism, a press-fit connector, hose/tube connector and/or the like, for securing the guide element at a desired distance relative to the patient site of interest.

In some embodiments, locking cap is screwed onto the additional guide element. Additional or alternative locking mechanisms can be brought to use for the prevention of medical device movement with respect to fixation base inside the guide channel and within the patient body.

A position for the operable coupling of the access port assembly external with or to the patient body may be determined in accordance with one or more clinical operational requirements.

The access port assembly may include at least one fastener for operably coupling the fixation base to the patient body, ensuring stable coupling of the base to the patient body, within the range of clinically allowable tolerance of the region identified as the site of interest.

In some embodiments, the fasteners of the fixation base to the patient body may include, for example, screws and/or adhesives suitable for operable engagement with the patient body.

In some embodiments, the orientation of the guide element may be manually manipulated into a desired trajectory. Alternatively, an automated or semi-automated access port manipulation system (not shown) can be used to control the orientation of guide element. Such access port manipulation system may be a robotic control system.

In some embodiments, such access port manipulation system may allow remote manipulation of the access port assembly for setting the orientation and position of the medical device relative to the patient body.

In some examples, the access port manipulation system may include or employ a virtual reality display system, a mixed-reality display system, or an augmented reality display system.

In some embodiments, a clinical authority (e.g., medical professional such as a physician) may confirm that the orientation of the guide element is secured in a safe and desired trajectory relative to a reference frame and/or that the medical device is secured at a desired distance relative to a patient site of interest, e.g., internal to the patient body. Additionally, or alternatively, access port assembly may be configured for setting the desired orientation and/or the desired distance of the medical device (or of the guide element) automatically or semi-automatically, with the assistance of or by a computerized platform.

While the fixation base is secured by fasteners to the patient body, and the guide element is secured by one or more lock mechanisms, safe access into the patient body can be ensured using suitable operational tools.

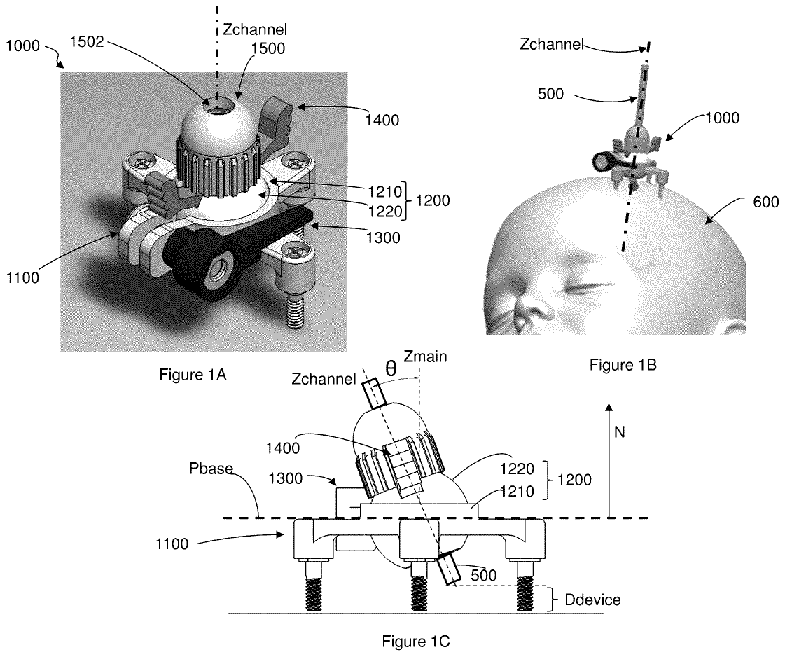

Reference is now made to FIGS. 1A-1C. According to some embodiments, an access port assembly 1000 is configured for fixating a medical device 500 in a desired orientation for deployment of medical 500 along a corresponding trajectory relative to a reference frame (e.g., head of a patient 600) and, optionally, for restricting movement of the medical device 500, once positioned at a desired position, along a guide axis Zguide of a guide element of access port assembly 1000.

According to some embodiments, elements of access port assembly 1000 may include a fixation base 1100, an articulating linkage 1200, an orientation lock mechanism 1300, a guide element 1400, and, optionally, a locking cap 1500.

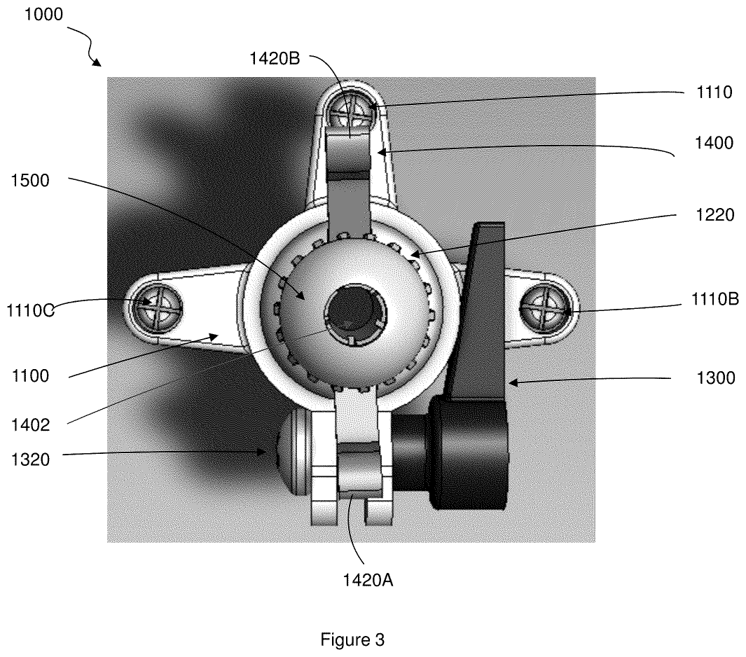

As will be outlined in further detail below, orientation lock mechanism 1300 may be employed for securing medical device 500 in a desired orientation relative to a reference frame (e.g., head of patient 600). In some embodiments, locking cap 1500, which comprises a cap bore 1502, may be employed for securing medical device 500 at a desired position along guide axis Zguide of a guide element (e.g., guide element 1400). This way, medical device 500 can be securely deployed at a desired angular orientation relative to a site of interest internal to patient body. Optionally, medical device 500 can be secured at a desired position (e.g., distance) relative to the patient site of interest internal to the patient body. When articulating linkage 1200, guide element 1400 and locking cap 1500 are operably engaged with each other, e.g., as shown in FIG. 3, the axes of their respective openings or channels may coincide with each other, allow inserting a medical device into the channel of guide element 1400. It is noted that the term “coincides”, as well as grammatical variations thereof, also encompasses the meaning of the term “substantially coincides”.

Guide element 1400 comprises a guide bore 1402 is configured to securely accommodate medical device 500, e.g., as outlined herein in more detail.

Articulating linkage 1200 of access port assembly 1000 may include a first articulating member or element 1210 and a second articulating member or element 1220 operably coupled with the first articulating member 1210 for forming the articulating linkage 1200. The terms “member” and “element” may herein be used interchangeably.

The first articulating member 1210 (e.g., a socket with a main axis Zmain and having a socket body or frame) may be configured to cooperatively receive the second articulating member (e.g., a ball) 1220, for example, for forming a ball-and-socket linkage. FIG. 1C schematically shows a configuration where the channel axis Zchannel of ball 1220 is tilted by an angle θ relative to the socket's main axis Zmain. The direction of main axis Zmain may be identical to the Normal N of a virtual base Pbase.

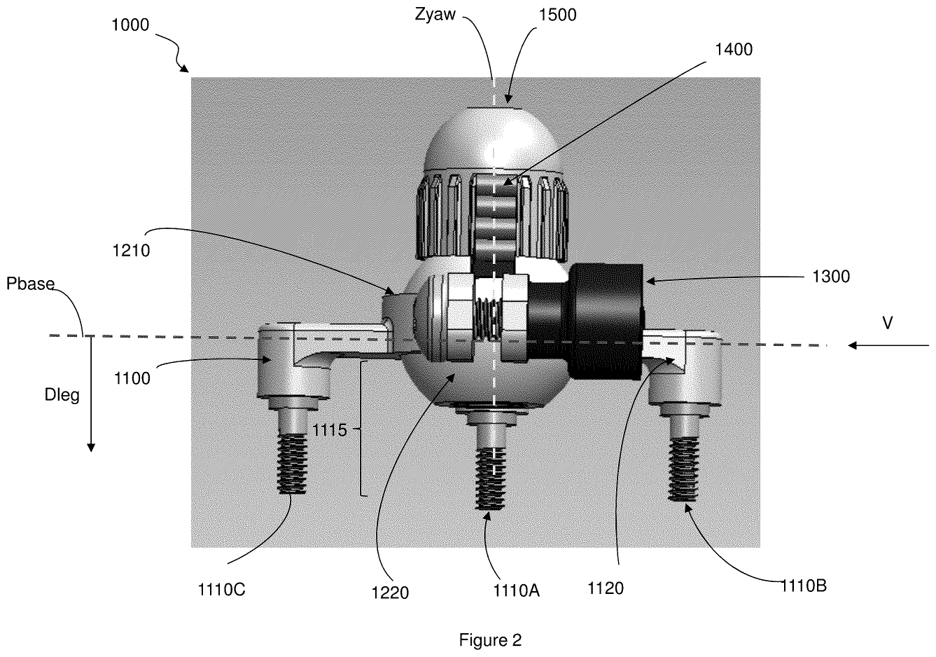

Additional reference is made to FIGS. 2-4. According to some embodiments, fixation base 1100 may include a frame structure 1120 that lies within or defines virtual base plane Pbase. In some examples, frame structure 1120 may be configured to include, form or constitute the first articulating member 1210.

Fixation base 1100 may be operably coupled to a part of the body of patient 600 by a plurality of fasteners 1110 proximally extending from frame structure 1120 towards the body of patient 600, e.g., for operable engagement with the patient's cranium.

Fasteners 1110 may be implemented by three or more legs that may extend proximally in direction Dleg from base plane Pbase. The three or more legs are herein exemplified as legs 1110A-C. In some examples, the three legs 1110A-C are arranged as vertices of an e.g., substantially equilateral or substantially isosceles triangle (FIG. 3). Alternatively, the plurality of fasteners 1110 may be arranged to delineate the vertices of any other polygon-shaped form, e.g., hexagon, pentagon, or rectangle. The fasteners may be implemented as cranial bolts.

In some embodiments, fixation base 1100, when operably engaged with the patient's cranium by fasteners 1110, is “floating” over the cranium, i.e., the entirety of fixation base 1100 is configured to provide a clearance gap Cgap 1115 between the cranium and the underside of fixation base 1100 facing the cranium. With respect to fixation base 1100, second articulating member 1220 may have proximal (or underside) portion and a distal (or upper) portion. When operably engaged with the cranium, the proximal portion of articulating member 1220 is located between the underside of fixation base 1100 and the cranium, and the distal portion of articulating member 1220 distally extends from the upper side of fixation base 1100.

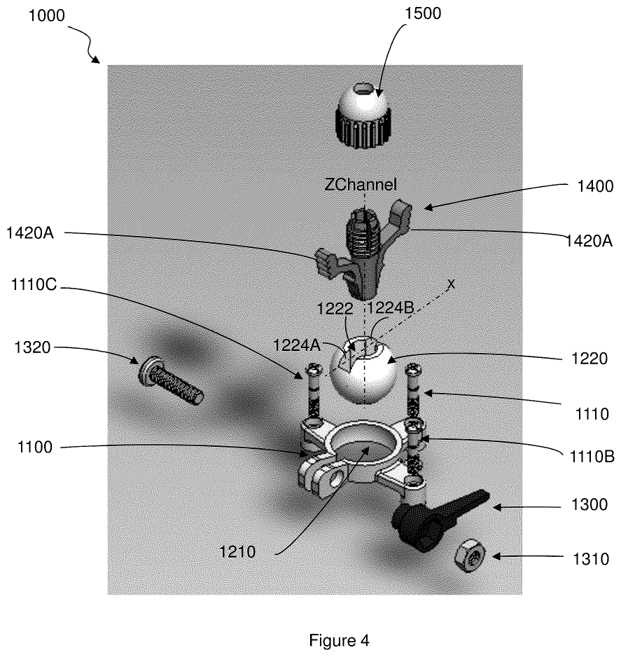

Clearance gap Cgap 1115 allows for an unobstructed line-of-sight (LOS) of the proximal portion of second articulating member 1220 when observed from a lateral viewing direction, e.g., as schematically indicated by arrow V. Depending on the yaw angle of viewing direction V with respect to yaw axis Zyaw, the LOS for viewing the proximal portion of articulating member 1220 may only be obstructed by the one or more legs 1110. Accordingly, a user and/or an imaging system may be able to view the orientation of the proximal portion of articulating member 1220 relative to the cranium, and allows viewing a distance Ddevice formed between a proximal end of medical device 500 (e.g., a catheter) and the patient's cranium during deployment of device 500 with the assistance of access port assembly 1000 for intracranial patient engagement, as for example schematically illustrated in FIG. 1C. Articulating linkage 1200 is operable to receive and/or includes a guide element having a guide channel, for slidably receiving medical device 500. In some examples, guide element may be implemented as guide element 1400 (e.g., shown in FIG. 4) that is separate from second articulating member 1220. In some other examples, second articulating member 1220 may include or constitute a channel 1222 that may be configured as a guide element for medical device 500.

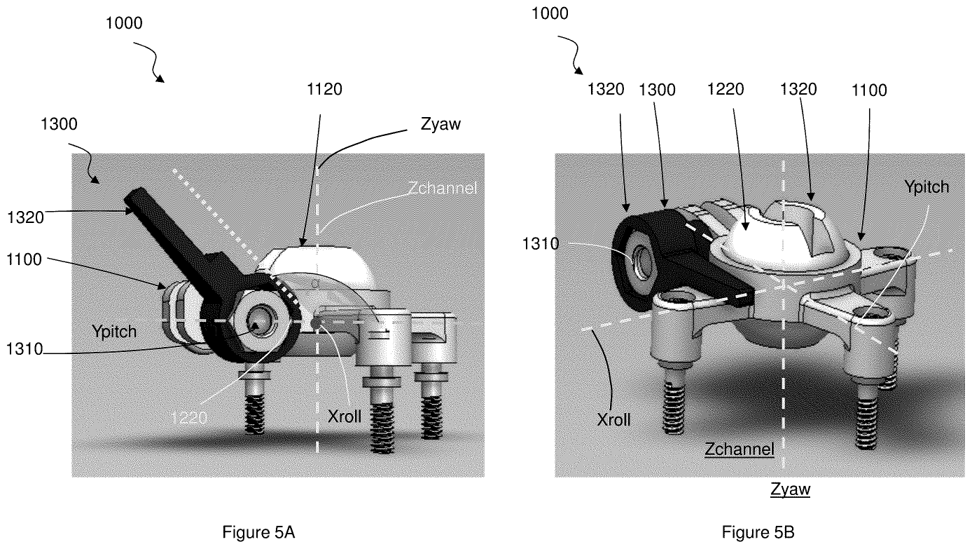

Additional reference is made to FIGS. 5A and 5B. As shown schematically in FIG. 5A, orientation lock mechanism 1300 is in an unlocked state such that ball 1220 is unsecured and thus free to rotate about roll axis Xroll, pitch axis Ypitch and yaw axis Zyaw. Conversely, once clinically desired orientation of Zguide is set, orientation lock mechanism 1300 is moved to the locked state shown in FIG. 3B, securing ball 1220 in a desired orientation, i.e., preventing ball 1220 from rotating about the axes Xroll, Ypitch and Zyaw.

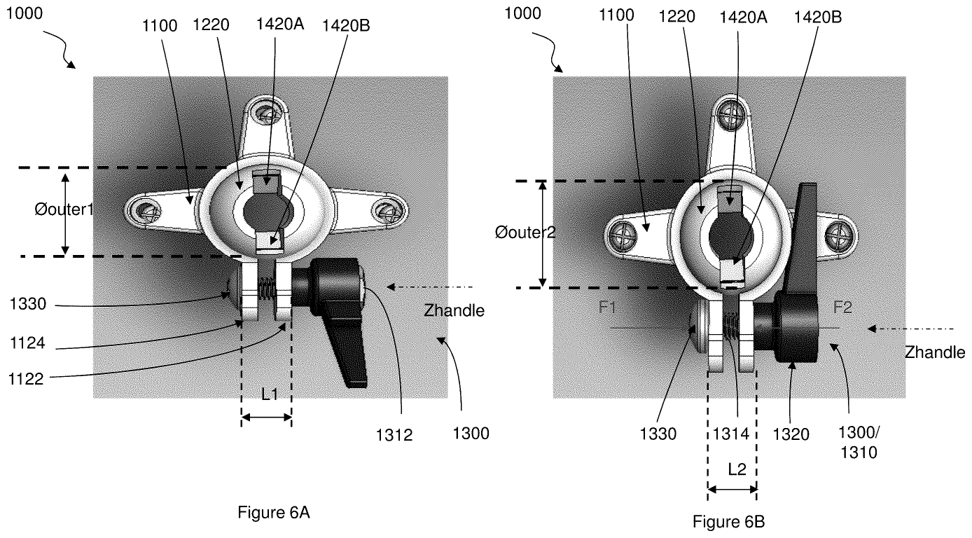

Further reference is made to FIGS. 6A and 6B. According to some embodiments, frame structure 1120 may be formed as C-shaped or U-shaped clamp extending from a first clamp end 1122 having a first hole end to an opposing second clamp end 1124 having a second hole opposite the first hole.

Orientation lock mechanism 1300 may include a bolt member 1310 having a head 1312, and an outer threaded portion 1314 that cooperates with an inner threaded portion (not shown) of a handle (also: lever) 1320 at one end where handle 1320 is sandwiched between first clamp end 1122 and bolt head 1312. Bolt member 1310 extends through the first clamp end 1122 and the second hole to terminate in a nut 1330 such both the first clamp end 1122 and the second clamp end 1124 are disposed between handle 1320 and nut 1330. In the illustrated example, turning handle (also: tilting lever) 1320 from the unlocked state (FIG. 6A) in clockwise direction relative to handle axis Zhandle about an angle a to attain the locked state (FIG. 6B), draws first clamp end 1122 and the second clamp end 1124 toward one another (arrows F1 and F2), thereby frictionally securing the orientation of ball 1220 relative to frame structure 1120. Configuration of orientation lock mechanism 1300 enables securing ball 1220 by tilting lever 1320 from a first position into a second position in a single lever engagement step. Clearly, additional, or alternative mechanisms for securing a fastener in place may be employed.

In the unlocked state (FIG. 6A), the inner surface parameter of fixation base 1100 has a diameter of Øouter1. After orientation lock mechanism 1300 is set into the locked state (FIG. 6B), the length of L is shortened from L1 to L2 due to bolt 1310 being locked into nut 1330 by bolt pitch increments, thereby obtaining diameter Øouter2<Øouter1.

Because the Øouter1 diameter of fixation base 1100 inner surface parameter is made smaller to Øouter2, pressure is applied onto the accommodated articulating member 1220, thus preventing rotation of articulating member 1220 about the axes Xroll, Ypitch and Zyaw, the frame structure thereby effectively functioning as a clamp mechanism. Alternatively, electrical, or electromechanical locking mechanisms may be employed.

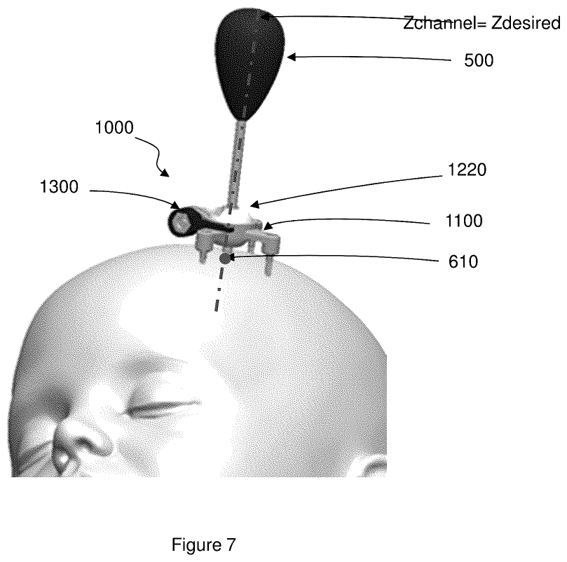

Further reference is made to FIG. 7. According to some embodiments, intracranial access may be gained by determining a clinically desired trajectory Zdesired by means of the articulating member or element 1220 accommodated by fixation base 1100, and by securing the articulating element in a locked state by orientation lock mechanism 1300 where the guide channel's axis Zchannel coincides with the desired trajectory Zdesired.

Point 611 designates the entry point (e.g., Kocher's point) into the patient body where a medical device (e.g., drill) shall penetrate the cranium. Medical device deployment trajectory coincides with the guide axis Zguide of the guide channel of, e.g., articulating element or member 1220.

According to some embodiments, after employing a drill device employed for drilling a hole into a patient's cranium, another medical device can be employed such as, for example, a biopsy needle, a catheter, a syringe, a flexible tube, a rigid tube, a guidewire, an expandable element and/or any instrument for gaining access to a site of interest internal to the patient boy, and/or for examining, and/or treating the patient.

Additionally, or alternatively, in some embodiments, articulating member 1220 may include an articulating device guide or articulating channel 1222 that may be configured to function as guide for safely executing medical procedure requiring the access to patient body in a predetermined trajectory. Articulating channel 1222 may, in some embodiments, be configured to receive a medical device 500 without requiring the employment of an additional guide element 1400.

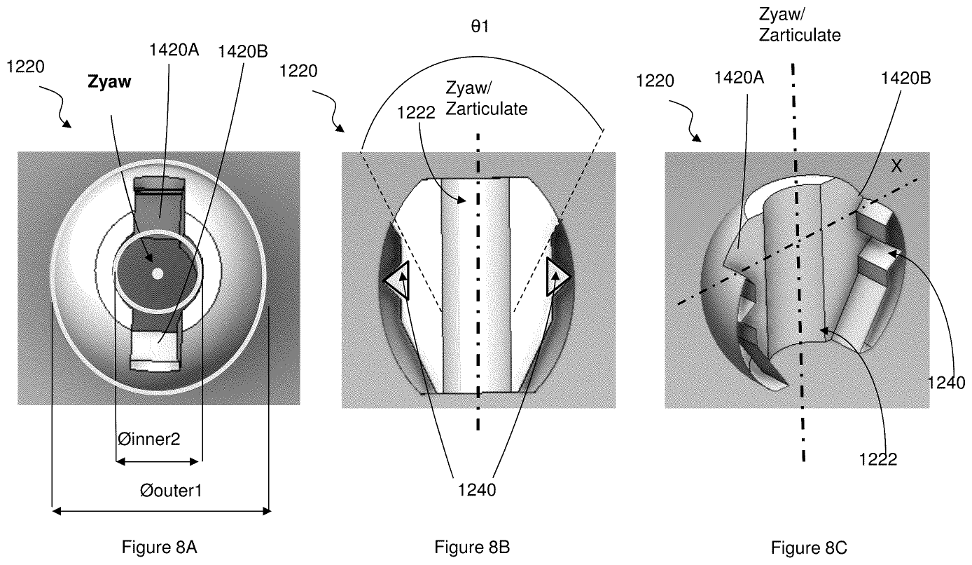

Reference is now made to FIG. 8A. In some embodiments, the articulating member 1220 has an outer diameter of Øouter1 permitting form-fittingly coupling the articulating element with fixation base 1100 to allowing rotation of articulating member 1220 about Xroll, Ypitch and Zyaw to set a guide element (e.g., element 1400) into a desired orientation relative to a reference frame.

In some embodiments, the inner diameter Øinner2 of the articulating member 1220 permits the passage of medical device 500 and/or guide element 1400 in the range of clear fit to frictional fit.

Additional reference is made to FIGS. 8B and 8C. According to some embodiments, FIG. 8B illustrates possible tilting angle range 01 about Xroll and Ypitch. In some examples, the rotation angles ranges about Xroll, Ypitch and Zyaw may differ from each other.

According to some embodiments, FIGS. 8B and 8C disclose stopper members 1240 radially protruding inwardly with respect to the axis Zarticulate a bore or channel 1222 of articulating member 1220. In some embodiments, the stopper members 1240 may define a passage that is tapering in proximal direction, to assist in the coupling of guide element 1400 with articulating member 1220 such that proximal movement of guide element 1400 towards the patient site of interest is form-fittingly limited and, optionally, frictionally limited. Stopper element (e.g., shoulder(s) or protrusion(s)) 1450 of guide element 1400 may be configured to cooperatively engage with corresponding stopper elements 1240 (e.g., groove(s) or recess(es)). In some implementations, stopper members 1240 may be configured such that guide element 1400 and/or medical device 500 are press-fit coupled with articulating member 1220.

The (e.g., tapering) passage may have various geometrical shapes and is not limited to the shape schematically illustrated in the accompanying figures. Accordingly, any shapes may be conceivable that allow, for example, to delimit proximal movement of the guide element and/or the medical device; and/or to maintain sufficient friction to achieve press-fit coupling of first articulating element with the mounted guide element and/or the medical device.

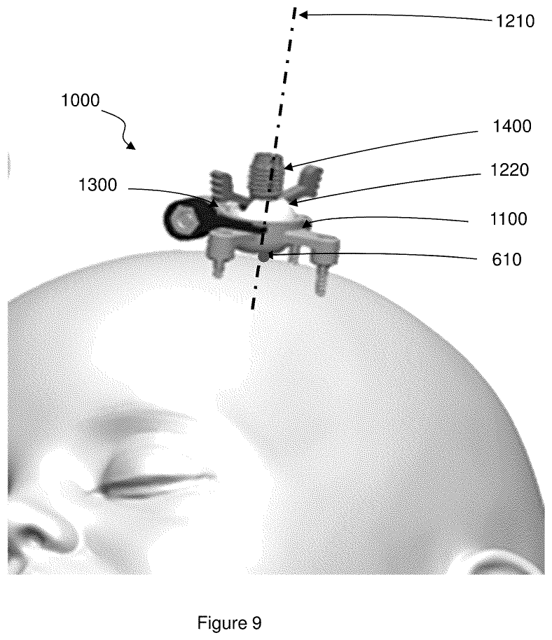

Further reference made to FIG. 9. According to some embodiments, guide element 1400 can be operably coupled with articulating member 1220. Articulating member 1220 is secured in a locked state by orientation lock mechanism 1300 for maintaining a consistent desired orientation of Zchannel to coincide with Zdesired relative to a reference frame.

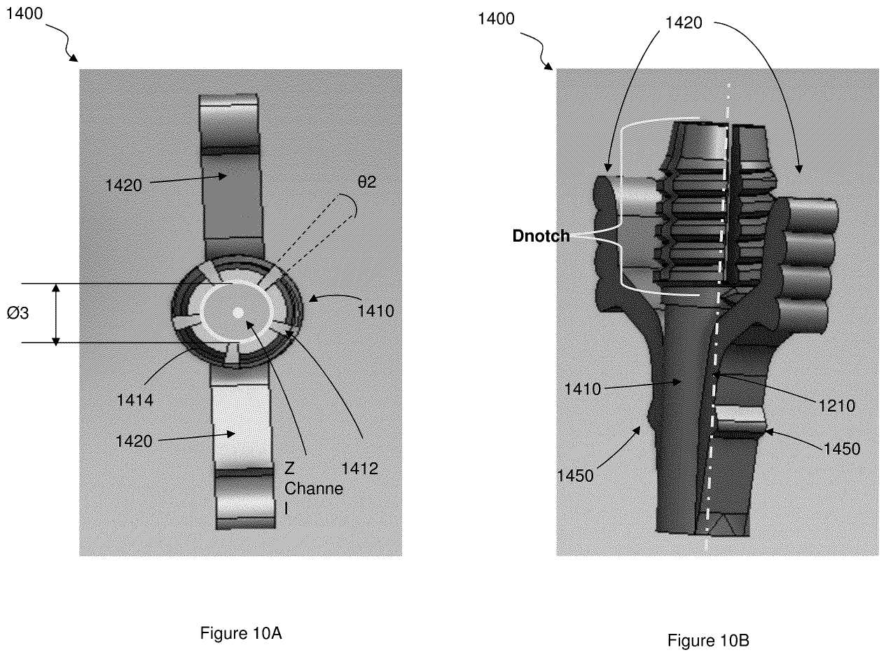

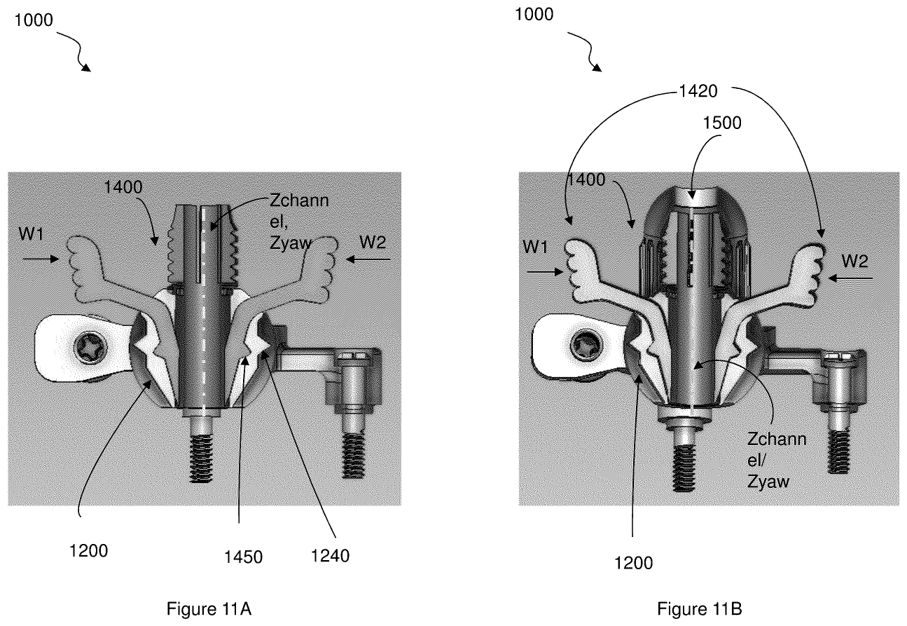

Reference is now made to FIG. 10A. In some embodiments, the guide element 1400 has a sleeve shaped base 1410 with an inner through hole diameter of Øinner3, of e.g., guide channel, permitting the insertion of a medical device, (e.g., catheter) into sleeve shaped base 1410, in the range of non-frictional to frictional fit clearance.

At least a part of sleeve shaped base 1410 may be threaded. In some embodiments, at least a portion of guide element thread may have slits or notches 1412 longitudinally extending, e.g., in parallel to the Zchannel, to create a plurality of elastically clampable and circumferentially arranged arch-shaped and deflectable threaded wings 1414 formed by a extending from sleeve shaped base 1410. Threaded wings 1414 may be at least partially exteriorly threaded.

Angle θ2 defines the angular width of each notch in the thread. The number of notches and angle of opening between two adjacent threaded wings 1414 with respect to center of guide element 1400 may vary.

Additional reference is made to FIG. 10B. In some embodiments, the depth of notch in the thread Dnotch may vary, e.g., for a same guide element or for different guide elements 1400.

In some embodiments, at least portion of the deflectable wings delineate an outer circumference that is larger than the inside circumference of the locking part to cause the clamping and threaded wings 1414 to be pressed inwardly by the inside thread of the locking part in direction towards the channel guide axis Zchannel.

In some embodiments, guide element 1400 may include a plurality of wing-shaped handles 1420 (also: handles 1420A and 1420B) suitable and configured for grabbing and manipulating, e.g., rotating relative to and/or deploying (e.g., pushing or pressing) the guide element 1400 into articulating channel 1222 of articulating member 1220.

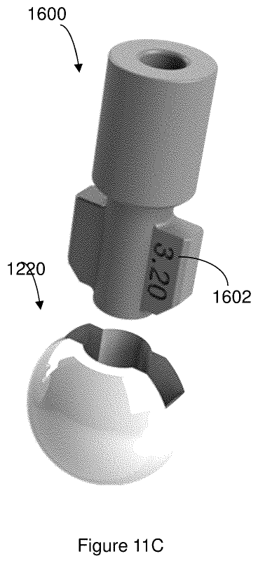

Further reference is made to FIG. 11A. According to some embodiments, cross-sectional view of the guide element 1400 shows a snug-fit engagement between stopper members 1240 and corresponding portions of guide element 1400. Consequently, guide element 1400 can be form-fittingly coupled with articulating linkage 1200, at least with respect to movement along their coinciding longitudinal axes and/or about their coinciding longitudinal axes.

In some embodiments, a guide element may be freely insertable and removable such to be form-fittingly coupled with respect to its rotation about the longitudinal axes Zchannel within second articulating member 1220 such that rotation of the guide element causes rotation of the second articulating member 1220, while the guide element can be pulled out from the second articulating member, without requiring unsecuring a fastener.

In some embodiments, a guide element may be deployable in second articulating member 1220 such that, once deployed, allows free rotation of the guide element within second articulating member 1220 about the Zchannel axis, while insertion and removal of the guide element requires securing and unsecuring a fastener.

Guide element 1400 and articulating channel 1222 are configured to realize a quick-release or quick-connect fitting mechanism, e.g., as described herein. By pressing wing-shaped handles 1420 against each other, as schematically illustrated with arrows W1 and W2, shoulders 1450 disengage from within recesses 1240, allowing pulling out guide element 1400 from second articulating member 1220 in longitudinal direction, schematically illustrated by arrow Pout. Guide element 1400 may be deployed through insertion (in direction of arrow Pin) into articulating linkage 1200 until shoulders 1450 snap-fit into grooves 1240. Guide element 1400 can be pulled out and inserted into second articulating member 1220 while the second articulating member is secured in a desired orientation relative to the cranium.

It is noted that, in some alternative embodiments, guide element 1400 can include grooves, and the articulating linkage can include shoulders or cooperating protrusions configured to cooperate with the grooves of the guide element, for realizing, e.g., a quick-release mechanism (e.g., a snap-fastener or connector). Additional, or alternative quick-connect mechanisms may be employed.

FIG. 11B depicts a cross sectional view of the access port assembly with a locking cap 1500 operably engaged with a portion of the plurality of elastically clampable and circumferentially arranged arch-shaped and deflectable threaded wings 1414 such that the threaded wings 1414 are pushed radially inwardly towards Zchannel to apply a frictional force onto a medical device (not shown) deployed in the guide channel. This way, the medical device is frictionally secured in a desired position along Zchannel.

The quick-release and quick connect mechanism realized by articulating linkage 1200 and guide element 1400, allows disengaging a medical device 500 from second articulating member 1220 by pressing wing-shaped handles 1420 against each other for decoupling guide element 1400 from articulating linkage 1200 to pull out guide element 1400 from second articulating member 1220.

The expressions “quick-release mechanism” and/or “quick-connect mechanism” may herein be used interchangeably, and either expression confers the meaning of “quick-connect” and “quick-release”.

In some embodiments, access port assembly 1000 may be configured such that wing-shaped handles 1420 can be pressed against each other for removal of guide element 1400 while locking cap 1500 is operably deployed.

In some embodiments, access port assembly 1000 may be configured such that wing-shaped handles 1420 can be pressed against each other for removal of guide element 1400 after removal of locking cap 1500.

In some embodiments, access port assembly 1000 may be configured such that wing-shaped handles 1420 can be pressed against each other for removal of guide element 1400 only after removal of locking cap 1500 from guide element 1400.

Further reference is now made to FIG. 11C, schematically showing a guide element 1600 in alignment relative to second articulating member 1220 of articulating linkage 1200, according to some alternative embodiments.

Accordingly, guide elements, as well as any of the other elements referred to herein, may come in different shapes and sizes. For example, guide element 1600 may not be equipped with wing-shaped handles and may be free of a thread for threadably receiving a locking cap. A guide element may be configured to be freely guided into and disengaged from the second articulating member 1220, without requiring operably engaging a quick-release mechanism to allow removal of the guide element. The same second articulating member 1220 can be configured to exchangeably receive guide elements of various designs. For instance, guide element 1600, which may be a drill guide, may have various internal drill guide diameters, suitable for use with corresponding drills inserts or drill bit diameters. Information about a drill bit diameter may be provided on the guide element, exemplified herein by annotation 1602 indicating a drill bit diameter of “3.20” mm.

Reverting to FIG. 3 and FIG. 4, second articulating member (e.g., ball) 1220 may include two recesses 1224A (show) and 1224B that laterally extend with respect to Zchannel along a second axis X, perpendicular to Zchannel. Recesses 1224A and 1224B are each configured to form-fittingly and/or snugly receive a portion of respective wing-shaped shaped handles 1420A and 1420B. Accordingly, rotating wing-shaped handles 1420 about axis Zchannel causes rotation of second articulating member 1220 and of medical device 500 accommodated by articulating linkage 1200 to rotate about axis Zchannel.

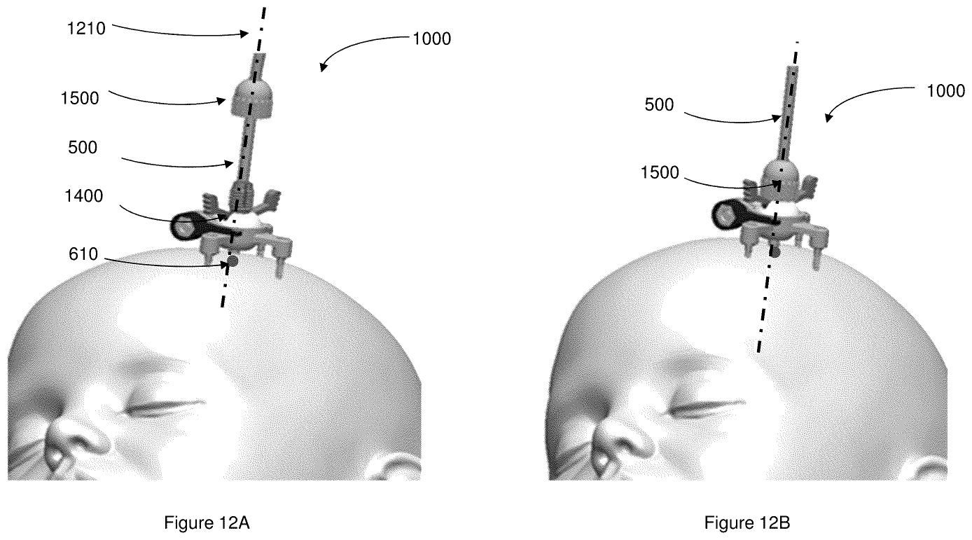

Rotating wing-shaped handles 1420 allows adapting the yaw-position of medical device about the axis Zchannel. Furthermore, wing-shaped handles 1420 may also facilitate adjusting the roll and pitch orientation of medical device 500 accommodated by articulating linkage 1200 relative to fixation base 1100 and, therefore, relative to the patient's cranium, while the fixation base 1100 is operably engaged with the cranium. Further reference is made to FIG. 12A. The coupling of guide element 1400 on top the of the second fixed articulating member 1220 is illustrated schematically, where medical device 500, e.g., a catheter, is inserted through the guide channel, without any vertical limitation, along the access axis Zchannel, on the catheter movement.

Further reference is made to FIG. 12B. Guide element 1400 may be coupled with (or accommodated by) second fixed articulating member 1220. A medical device 500, e.g., a catheter, may be inserted through the guide channel of guide element 1400, after which a locking cap 1500 may be cooperatively threaded onto the guide element 1400 for frictionally limiting vertical movement of medical device 500 along the access axis Zchannel.

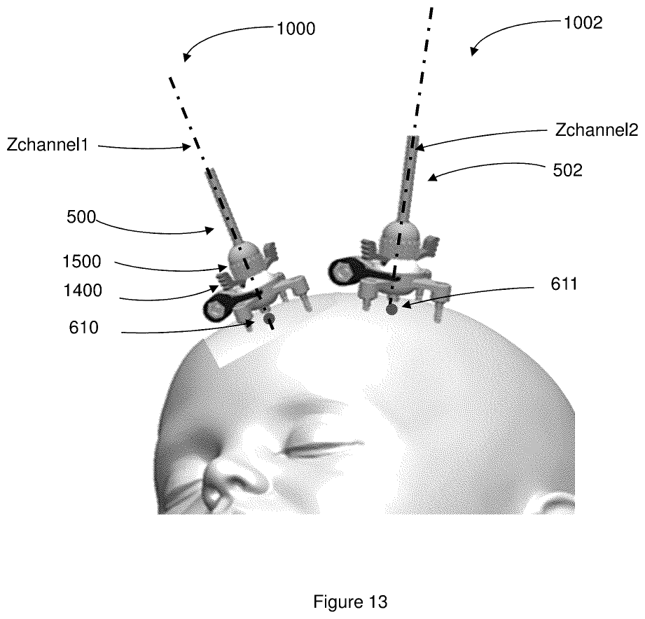

Reference is now made to FIG. 13. According to some embodiments, a plurality of access port assemblies 1000 and 1002 may be employed. In such case, at least two different point of entry are marked, 610 and 611, each optionally having a distinct trajectory axis Zchannel1 and Z channel2 for the deployment of a first medical device 500 and a second medical device 502, respectively, in operable conjunction with an inlet access port assembly 1000 and an outlet access port assembly 1002.

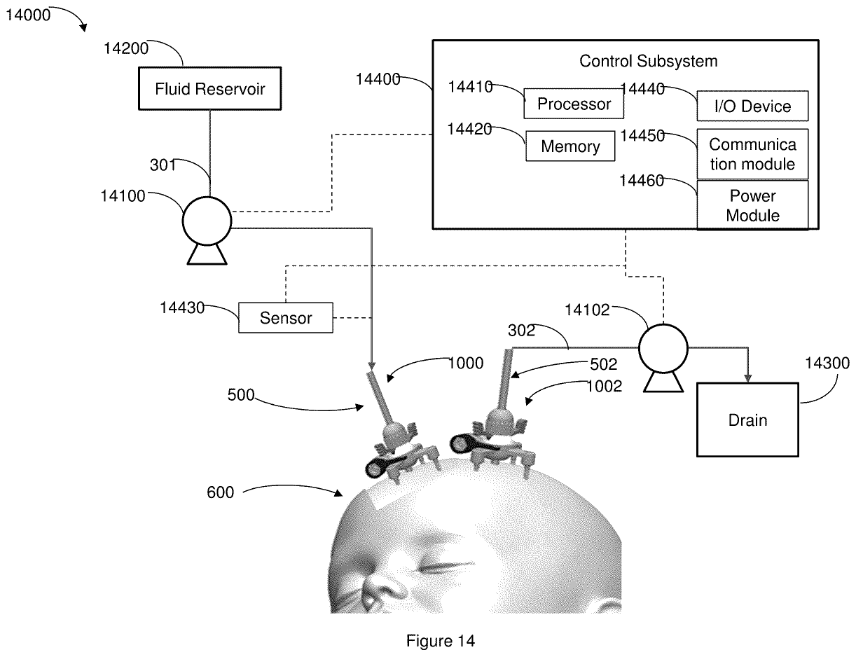

Reference is now made to FIG. 14. According to some embodiments, a flow control system 14000 is configured to control fluid input into and/or output from the patient's body, assisted by one or more pumps, via one or more access port assemblies. In some examples, fluid output may flow spontaneously out of the patient's body. In some examples, a single port and a pump may be employed for controlled delivery into and/or aspiration of fluid from within the patient body.

In some embodiments, fluid 301 approved for use inside the patient body may be delivered by a first pump 14100 from a fluid reservoir 14200 to one or more patients. In some embodiments, fluid output may be actively controlled by a second pump 14102, for example, to relieve intracranial pressure. In some examples, excess intracranial pressure may be controllably drained to drain 14300 without the employment of a second pump 14102, e.g., by gravitation. In some examples, input may be provided based on gravity (e.g., Buretrol), an output pump 14102 for drain. In some examples, a direction of fluid flow may be reversed so that first pump 14100 functions as an outlet pump and second pump 14102 functions as an inlet pump.

In some examples, system 14000 may be configured to create fluid circulation with a net fluid output of zero, e.g., for blood clot removal, reducing inflammation, and/or for other treatments.

Fluid 301 may for example be clinically appropriate for the treatment of a patient, e.g., ensuring safe inner pressure values. In addition, or alternatively, pharmaceutical active agent may be delivered via an input access port assembly such as antibiotic agents, blood thinners, anti-inflammatory agents, chemotherapy agents, immunotherapy agents, or any combination of the aforesaid.

In some examples, chemotherapy and/or other therapeutic agents may be controllably delivered at increased pressure, above normal intracranial pressure, for a limited period, but such that the agents can reach deep brain tissue, e.g., for the treatment of (e.g., deep brain tissue) affected by malignant tumors.

In some embodiments, removal of fluid via an access port assembly allows for the drainage of biological fluids, e.g., cerebral spinal fluid (CSF) 302. In some examples, drainage may include controllably draining fluids to relieve elevated intracranial pressure. Optionally, flow control system 14000 may include at least one valve (not shown) for controllably draining fluids. Controllably draining fluids may include controlling the at least one valve to divert, via an outlet access port assembly 1002, output or waste fluid to drain, e.g., for the removal of blood clots.

In some examples, flow control system 14000 may be man-portable (also: mobile), easy-to-use and employable in a variety of ambulatory or outpatient care settings such as in a domestic environment and/or while the patient is mobilized or during transportation.

In some examples, parts or all of flow control system may be configured as a cartridge. The system may be configured to provide sterile fluid that suffices for a single exchange, batch or CSF drainage cycle.

In some embodiments, capacity of reservoir 14200 reservoir capacity may be in the range of, for example, 250-750 milliliters.

In some embodiments, flow control system 14000 may be operable to deliver a predetermined amount of fresh sterile fluid.

In some embodiments, flow control system 1400 can be used to analyze the removed fluid (e.g., for electrolyte level, chloride concentration and/or any other analytes).

In some other embodiments, the flow control system may be operable to provide a variable amount of fresh sterile fluid, e.g., depending on the patient's needs.

Optionally, at least some or all components of flow control system 14000 may be configured for single use (e.g., disposable). Optionally, at least some or all components of flow control system 14000 may be configured as a cartridge. Optionally, a flow control system may comprise a housing (e.g., of an exchangeable cartridge configuration) enclosing some or all system's components.

At least some or all components of flow control system 14000 may be configured for single use. At least some or all components of the system may be disposable.

In some embodiments, flow control system 14000 may be configured to be entirely mechanically operable, i.e., without requiring any electrical power supply such as a battery and/or connection to an electrical grid. Optionally, the flow control system 14000 may be selectively mechanically, at least partially or fully automatically operable.

In some embodiments, flow control system 14000 may require electrical power supply such as a battery, to allow for automated sensing functionalities of system operating parameters and related controls, as will be outlined herein in more detail.

According to some embodiments, flow control system 14000 includes at least one sensor 14430 configured to provide at least one sensor output relating to at least one sensed characteristic of cerebrospinal fluid pressure.

According to some embodiments, the at least one sensed characteristic includes one of the following: fluid flow rate, fluid pressure, fluid temperature, concentration of one more target analyte in the fluid, or any combination of the aforesaid.

According to some embodiments, flow control system 14000 comprises a control subsystem 14400 that includes at least one processor 14410 and at least one memory 14420 storing data and software code portions executable by the at least one processor to cause the system to receive the at least one input from a sensor 14430; and to control, based on the at least one sensor input, the operation of various components of the system.

In some embodiments, insertion and/or exertion fluid flow rate can be controlled by predetermined by at least one liquid characteristic threshold, which includes: cerebral pressure, which may pertain to intracranial pressure, and/or the cerebrospinal fluid (CSF) pressure.

According to some embodiments, multiple physiological parameters can be tracked by flow control system 14000. Physiological parameters can include one of the following: systolic blood pressure, diastolic blood pressure, mean arterial pressure, pulse rate, breathing rate, breathing pattern, oxygen saturation level, glucose level, an electrical property of skin, weight, body-mass index, pH level, concentration of one or more selected analytes in bodily fluid, motor function, body temperature, sweat rate, electrocardiogram, myocardiogram, electroencephalography, capnography values, cognitive ability, drained CSF (e.g., chemical) analysis or any combination of the aforesaid.

In some embodiments, flow control system, further comprises: a fluid reservoir 14200 that is in fluid communication with the at least one pump device for controllably providing the patient with fluid stored in the fluid reservoir 14200 via an inlet access port assembly 1000.

In some embodiments of flow control system 14000, the fluid that can be stored in reservoir 14200 can include or can contain fresh liquid for washout of (e.g., infected) fluid, excess fluid, liquid containing at least one pharmaceutically active agent, or any combination of the aforesaid.

The term “processor”, as used herein, may additionally or alternatively refer to a controller. Processor 14410 may be implemented by various types of processor devices and/or processor architectures including, for example, embedded processors, communication processors, graphics processing unit (GPU)-accelerated computing, soft-core processors and/or general-purpose processors.

Memory 14420 may be implemented by various types of memories, including transactional memory and/or long-term storage memory facilities and may function as file storage, document storage, program storage, or as a working memory. The latter may for example be in the form of a static random-access memory (SRAM), dynamic random-access memory (DRAM), read-only memory (ROM), cache and/or flash memory.

As working memory, memory 14420 may, for example, include, e.g., temporally-based and/or non-temporally based instructions. As long-term memory, memory 14420 may for example include a volatile or non-volatile computer storage medium, a hard disk drive, a solid-state drive, a magnetic storage medium, a flash memory and/or other storage facility. A hardware memory facility may for example store a fixed information set (e.g., software code) including, but not limited to, a file, program, application, source code, object code, data, and/or the like.

Subsystem 14400 may further include an input/output device 14440 which may be configured to provide or receive any type of data or information. input/output device 14440 may include, for example, visual presentation devices or systems such as, for example, computer screen(s), head mounted display (HMD) device(s), first person view (FPV) display device(s), device interfaces (e.g., a Universal Serial Bus interface), and/or audio output device(s) such as, for example, speaker(s) and/or earphones. Input/output device 14440 may be employed to access information generated by the system and/or to provide inputs including, for instance, control commands, operating parameters, queries and/or the like. For example, input/output device 14440 may allow a user of flow control subsystem 14400 to perform one or more of the following: activating or deactivating pump operation, increasing or decreasing pump speed, increasing or decreasing flow rate, increasing or decreasing valve opening, and/or the like.

Subsystem 14400 may further comprise at least one communication module 14450 configured to enable wired and/or wireless communication between the various components and/or modules of the system and which may communicate with each other over one or more communication buses (not shown), signal lines (not shown) and/or a network infrastructure.

RF-based wireless communication; optical-based wireless communication such as infrared (IR) based signaling, and/or wired communication. The network may be configured for using one or more communication formats, protocols and/or technologies such as, for example, to internet communication, optical or RF communication, telephony-based communication technologies and/or the like. In some examples, communication module 14450 may include I/O device drivers (not shown) and network interface drivers (not shown) for enabling the transmission and/or reception of data over the network. A device driver may, for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G, 5G, 6G mobile networks, 3GPP, LTE, LTE advanced, Bluetooth® (e.g., Bluetooth smart), ZigBee™, near-field communication (NFC) and/or any other current or future communication network, standard, and/or system.

Flow control system 14000 may further include a power module 14460 for powering the various components and/or modules and/or subsystems of the system. Power module 14460 may comprise an internal power supply (e.g., a rechargeable battery) and/or an interface for allowing connection to an external power supply.

It will be appreciated that separate hardware components such as processors and/or memories may be allocated to each component and/or module of flow control system 14000. However, for simplicity and without be construed in a limiting manner, the description and claims may refer to a single module and/or component. For example, although processor 14410 may be implemented by several processors, the following description will refer to processor 14410 as the component that conducts all the necessary processing functions of system 14000.

Functionalities of system 14000 may be implemented fully or partially by a multifunction mobile communication device also known as “smartphone”, a mobile or portable device, a non-mobile or non-portable device, a digital video camera, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a non-vehicular device, a stationary device and/or a home appliances control system.

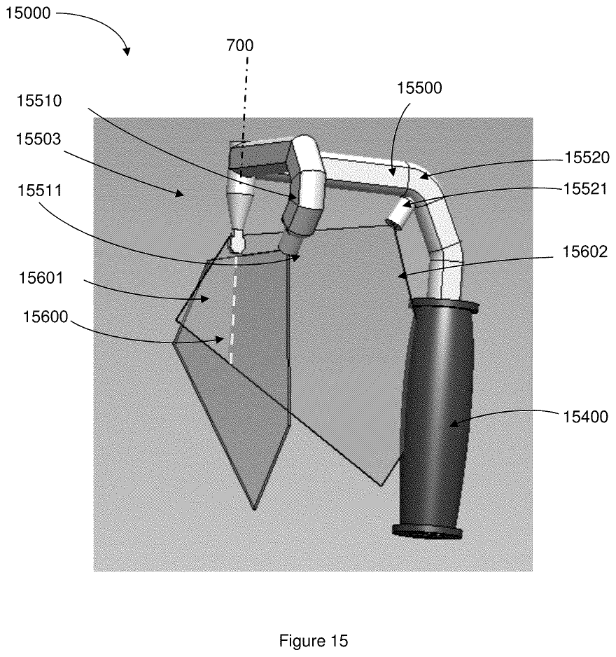

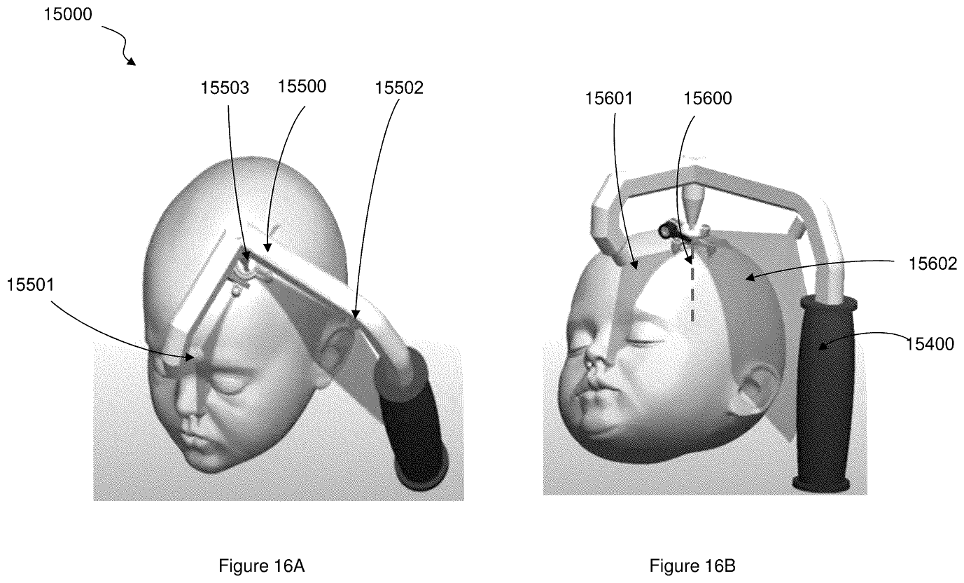

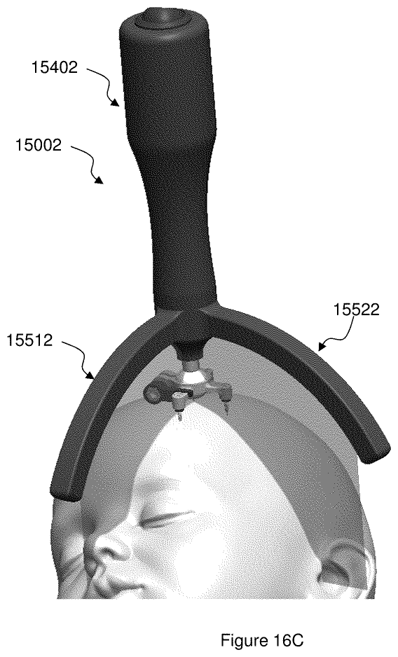

Reference is now made to FIG. 15 and to FIGS. 16A and 16B. A trajectory determination assembly 15000 is shown which is configured for assisting in determining a desired trajectory of a ventricular drainage catheter through a cranial target site identified via the Kocher's point, and/or through imaging (e.g., CT, MRI), and/or any other anatomical landmarks clinically suitable for proceeding operation.

According to some embodiments, the trajectory determination assembly 15000 comprises a first arm 15510 and a second arm 15520 that are articulated with each other to enable rotation relative to each other around a rotation axis 700. One of the arms (e.g., second arm 15520) may proximally extend (in direction of the patient) into a handle 15400.

According to some embodiments, the first arm 15510 comprises a first light source 15511 configured to emit in a first projection orientation, which is selectable with respect to at least one first facial feature, a first projection plane 15601 to project a first line marking on the patient's head.

According to some embodiments, the second arm 15520 comprises a second light source 15521 configured to emit in a second projection orientation, which is selectable with respect to at least one second facial feature, a second projection plane 15602 to project a second line marking on the patient's head.

According to some embodiments, the trajectory determination assembly 15000 is configured such that when the first arm 15510 and the second arm 15520 are concurrently set, respectively, in the selected first and second projection orientations, the rotation axis 700 of the first arm and the second arm coincides with a desired catheter trajectory Zchannel.

According to some embodiments, first light source 15511 and second light source 15521 are configured with respect to first arm 15510 and second arm 15520 such that an intersection line 15600 between first projection plane 15601 and second projection plane 15602 coincides with the rotation axis 700.

In some embodiments, first arm 15510 and second arm 15520 are joined together by a pivot linkage 15503 that comprises a proximally extending shaft that is receivable by the guide of an access port assembly 1000.

In some embodiments, first light source 15511 and second line light source 15521 each have, respectively, a plane projection direction that is fixed relative to the first arm 15510 and the second arm 15520.

In some embodiments, the first 15511 and/or the second 15521 light sources of the trajectory determination assembly 15000 are laser light sources.

In some embodiments, the trajectory determination assembly is configured to be manually, semi-automatically or fully-automatically manipulatable for determining the desired medical device trajectory.

In some embodiments, the first and the second arms, when operably engaged, are positioned superior to the patient's cranium.

Referring now to FIG. 16C, a trajectory determination assembly 15002 is shown, according to some alternative embodiments. In the configuration shown, manipulating handle 15402 may distally extend from the intersection between a first arm 15512 and a second arm 15522. Analogous to what is described with respect to assembly 15000, assembly 15002 may comprise light sources for assisting in determining the location of an anatomical landmark, e.g., as outlined herein with respect to FIGS. 15 and 16A-16B.

### ADDITIONAL EXAMPLES

Example 1 pertains to an access port assembly configured to assist in creating access for and accommodating a medical device extending from outside a patient body and terminating in a cavity internal to the patient body, the access port assembly comprising:
a fixation base comprising at least one fastener for securely coupling the access port assembly in a selected position to a site of interest external to the patient body;
a guide element comprising a guide channel having a longitudinal guide axis extending from an inlet to an outlet of the guide element for guiding the medical device through the guide channel, wherein the guide element is movably coupled with the fixation base by an articulating linkage to allow selecting an orientation of the guide channel relative to the site of interest of the patient body;
an orientation locking mechanism configured to secure the guide channel in a selected orientation and to unsecure the guide channel to allow manipulation of the guide channel into a selected orientation relative to the fixation base;
a guide locking mechanism for axially securing the medical device in a desired position within the guide channel of the guide element, wherein the articulating linkage is implemented as a ball-and-socket linkage.
Example 2 includes the subject matter of Example 1 and, optionally, wherein the assembly is configured to assist in providing a burr hole in a patient's cranium and for fixating, relative to the patient's cranium, a catheter to be deployed via the burr hole to provide fluid communication between an intracranially located cavity and an extracranially located cavity.
Example 3 includes the subject matter of Example 1 and/or Example 2, and, optionally, access port assembly of Example 1 or Example 2, wherein the at least one fastener includes screws and/or adhesives.
Example 4 includes the subject matter of any one or more of the Examples 1 to 4 and, optionally, wherein the orientation locking mechanism is configured to frictionally and/or form-fittingly secure the guide channel in a selected orientation.
Example 5 includes the subject matter of access port assembly of any one or more of the Examples 1 to 4, and, optionally, wherein the guide element comprises one more handles for facilitating manipulation of the guide element into a desired orientation relative to the fixation base.
Example 6 includes the subject matter of any one or more of the Examples 1 to 6, and, optionally, wherein the guide element is tiltably coupled with the fixation base for tilting of the guide element around at least one tilt axis relative to the fixation base.
Example 7 includes the subject matter of any one or more of the Examples 1 to 7 and, optionally, wherein the guide element is tiltably coupled with the fixation base for tilting of the guide element relative to the fixation base around a plurality of different tilt axes including a first tilt axis X and a second tilt axis Y that is perpendicular to the first tilt axis X.
Example 8 includes the subject matter of any one or more of the Examples 1 to 8, and, optionally, wherein the guide element is rotatable around a pivot axis R that is perpendicular to the first tilt axis X and the second tilt axis Y.
Example 9 includes the subject matter of examples 7 or 8, and, optionally, wherein the first tilt axis X defines a roll axis, the second tilt axis Y defines a pitch axis, and the pivot axis R defines a Yaw axis relative to the fixation base.
Example 10 includes the subject matter of any one or more of the examples 1 to 9 and, optionally, comprising a linkage clamp mechanism configured to apply pressure onto the socket of the ball-and-socket joint for frictionally securing the guide channel in a selected orientation relative to the fixation base.
Example 11 includes the subject matter of example 10 and, optionally, wherein the linkage clamp mechanism comprises a locking mechanism.
Example 12 includes the subject matter of any one or more of the examples 1 to 11 and, optionally, wherein the guide element is configured to receive a medical device including one or more of the following: a drill, a catheter, a flexible tube, a rigid tube, a guidewire, or an expandable element.
Example 13 includes the subject matter of any one or more of the Examples 1 to 12 and, optionally, wherein the assembly is configured for guiding a drill to create a bore hole into a patient's cranium for the deployment of a catheter through the bore hole to perform one or more of the following: ventricular drainage; intracranially delivering a pharmaceutical substance, or a combination of the aforesaid.
Example 14 includes the subject matter of one or more of the examples 1 to 13 and, optionally, wherein the guide locking mechanism is configured to frictionally and/or form-fittingly secure the medical device in a desired position within the guide channel.
Example 15 includes the subject matter of any one or more of the examples 1 to 14 and, optionally, wherein the guide locking mechanism includes a guide clamp configured to radially apply pressure onto the external surface of a medical device received by the guide channel for frictionally securing the medical device in a selected axial position within the guide channel.
Example 16 includes the subject matter of any one or more of the examples 1 to 15 and, optionally, the guide locking mechanism comprises: a sleeve-shaped part having a longitudinal sleeve axis that coincides with the longitudinal channel guide axis, wherein the sleeve-shaped part comprises:
a base; and
a plurality of elastically clampable and circumferentially arranged arch-shaped and deflectable wings extending from the base, wherein the wings are at least partially exteriorly threaded; and
a locking ring comprising an interiorly threaded opening for cooperatively and lockingly engage with the exterior threads of the sleeve by radially applying with the wings pressure onto a medical device received in the guide channel.
Example 17 includes the subject matter of example 16, and optionally, wherein at least portion of the deflectable wings delineate an outer circumference that is larger than the inside circumference of the locking part to cause the clamping wings to be pressed inwardly by the inside thread of the locking part in direction towards the channel guide axis.
Example 18 includes the subject matter of any one or more of the Examples 1 to 17 and, optionally, comprising a locking cap that includes the locking ring.
Example 19 pertains to a system for monitoring and controlling flow of intracranial fluid, the system comprising: at least one first cranial inlet access port assembly,
at least one second cranial outlet access port assembly;
at least one pump device configured to impart energy onto fluid, and having a pump inlet and a pump outlet;
wherein the first inlet access port assembly and the pump outlet can be brought in fluid communication with each other by a cranial inlet catheter tubing; and
wherein the first outlet access port assembly and the pump inlet can be brought in fluid communication with each other by a cranial outlet catheter tubing;
at least one sensor configured to provide at least one sensor output relating to at least one sensed characteristic of cerebrospinal fluid pressure;
at least one processor;
at least one memory storing software code portions executable by the at least one processor to cause the system to perform the following steps:
receiving the at least one sensor output; and
controlling, based on the at least one sensor output, the operation of the at least one pump device.
Example 20 includes the subject matter of example 20 and, optionally, wherein the at least one sensed characteristic includes one of the following: fluid flow rate, fluid pressure, fluid temperature, concentration of one more target analytes in the fluid, or any combination of the aforesaid.
Example 21 includes the subject matter of example 20 and, optionally, wherein the at least one liquid characteristic includes: intracranial pressure, and/or cerebrospinal fluid (CSF) pressure.
Example 22 includes the subject matter of any one or more of the examples 19 to 21 and, optionally, a drainage tubing; and wherein the steps further include: controllably draining CSF to relieve elevated intracranial pressure.
Example 23 includes the subject matter of example 22 and, optionally, at least one valve, wherein controllably draining CSF includes controlling the at least one valve to divert intracranial fluid to drain and/or removing blood clots.
Example 24 includes the subject matter of example 22 and/or 23 and, optionally, wherein controllably draining CSF includes controlling the at least one pump to divert CSF to drain.
Example 25 includes the subject matter of any one or more of the preceding examples 19 to 24 and, optionally, a fluid reservoir that is in fluid communication with the at least one pump device for controllably providing the patient with fluid stored in the fluid reservoir via the first cranial inlet access port assembly.
Example 26 includes the subject matter of Example 25 and, optionally, wherein the fluid that can be stored in the reservoir can include or can contain: fresh liquid for washout of (e.g., infected) fluid, excess CSF fluid, liquid containing at least one pharmaceutically active agent, or any combination of the aforesaid.
Example 27 includes the subject matter of Example 26 and, optionally, wherein the at least one pharmaceutically active agent includes: antibiotic agents, blood thinners, anti-inflammatory agents, chemotherapy agents, immunotherapy agents, or any combination of the aforesaid.
Example 28 pertains to a trajectory determination assembly configured for assisting in determining a desired trajectory of a ventricular drainage catheter through a cranial target site, the trajectory determination assembly comprising:
a first arm and a second arm that are articulated with each other to enable rotation relative to each other around a rotation axis;
wherein the first arm comprises a first light source configured to emit in a first projection orientation, which is selectable with respect to at least one first facial feature, a first projection plane to project a first line marking on the patient's head;
wherein the second arm comprises a second light source configured to emit in a second projection orientation, which is selectable with respect to at least one second facial feature, a second projection plane to project a second line marking on the patient's head; and
wherein the trajectory guide is configured such that when the first arm and the second arm are concurrently set, respectively, in the selected first and second projection orientations, the rotation axis of the first arm and the second arm coincides with the desired catheter trajectory.
Example 29 includes the subject matter of example 28 and, optionally, wherein the first and the second light source are configured with respect to the first and the second arm such that an intersection between the first and the second projection plane coincides with the rotation axis.
Example 30 includes the subject matter of Examples 28 and/or 29 and, optionally, wherein the first and second arms are joined together by a pivot linkage that comprises a proximally extending shaft that is receivable by the guide of an access port assembly, e.g., of any one or more of the examples 1 to 19.
Example 31 includes the subject matter of Examples 28 to 30 and, optionally, wherein the first and the second line light sources each have, respectively, a plane projection direction that is fixed relative to the first and the second arm.
Example 32 includes the subject matter of any one or more of the Examples 28 to 31 and, optionally, wherein the first and/or the second light sources are laser lights.
Example 33 includes the subject matter of any one or more of the Examples 28 to 32 and, optionally, configured to be manually, semi-automatically or fully-automatically manipulatable for determining the desired catheter trajectory.
Example 34 includes the subject matter of any one or more of the Examples 28 to 33 and, optionally, wherein the first and the second arms, when operably engaged, are positioned superior to the patient's cranium.

In some examples, the fixation base, when operably engaged with the cranium, provides a clearance gap between the cranium and the underside of fixation base facing the cranium.

In some examples, the guide element is movably coupled with the fixation base by an articulating linkage to allow selecting an orientation of the guide channel relative to the site of interest of the patient body; and wherein the guide element and the articulating linkage are configured so that they are form-fittingly coupleable with each other.

In some examples, the guide element and the articulating linkage are configured as or configured to include a quick-connect mechanism allowing quick-connecting a medical device with the articulating linkage and quick-releasing a medical device from the articulating linkage. In some examples, the quick-release and quick-connect can be performed while the second articulating member is at any desired position relative to the cranium.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

Any digital computer system, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may directly loadable into an internal memory of a digital computer, comprising software code portions for performing the methods and/or processes as disclosed herein. The term “non-transitory” is used to exclude transitory, propagating signals, but to otherwise include any volatile or non-volatile computer memory technology suitable to the application.

Additionally, or alternatively, the methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, assemblies, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms ‘about’ and/or ‘close’ with respect to a magnitude or a numerical value may imply to be within an inclusive range of −10% to +10% of the respective magnitude or value.

“Coupled with” means indirectly or directly “coupled with”.

As used herein, the terms “proximal” and “distal” respectively, refer to a position closer to and away from a patient's body that would receive a port device.

The terms “longitudinal” and “axial” are interchangeable, and refer to an axis extending in the proximal and distal directions, unless otherwise expressly defined.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not to be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

As used herein the term “configuring” and/or ‘adapting’ for an objective, or a variation thereof, implies using materials and/or components in a manner designed for and/or implemented and/or operable or operative to achieve the objective.

Unless otherwise stated or applicable, the use of the expression “and/or” between the last two members of a list of options for selection indicates that a selection of one or more of the listed options is appropriate and may be made, and may be used interchangeably with the expressions “at least one of the following”, “any one of the following” or “one or more of the following”, followed by a listing of the various options.

As used herein, the phrase “A, B, C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A, B, C), but extends to fewer and greater numbers of elements (e.g., A, B, C, D, etc.).

It is noted that the terms “operable to” or “operative to” can encompass the meaning of the term “adapted or configured to”. In other words, a machine “operable to” or “operative to” perform a task can in some embodiments, embrace a mere capability (e.g., “adapted”) to perform the function and, in some other embodiments, a machine that is actually made (e.g., “configured”) to perform the function.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be appreciated that combination of features disclosed in different embodiments are also included within the scope of the present inventions.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An access port assembly configured to assist in creating access for and accommodating a medical device extending from outside a patient body and terminating in a cavity internal to the patient body, the access port assembly comprising:
a fixation base having and underside and comprising at least one fastener for securely coupling the access port assembly in a selected position to a site of interest external to the patient body;
a guide element comprising a guide channel having a longitudinal guide axis extending from an inlet to an outlet of the guide element for guiding the medical device through the guide channel,
wherein the guide element is movably coupled with the fixation base by an articulating linkage to allow selecting an orientation of the guide channel relative to the site of interest of the patient body;
an orientation locking mechanism configured to secure the guide channel in a selected orientation and to unsecure the guide channel to allow manipulation of the guide channel into a selected orientation relative to the fixation base;
a guide locking mechanism for axially securing the medical device in a desired position within the guide channel of the guide element,
wherein the articulating linkage is implemented as a ball-and-socket linkage; and
wherein the fixation base, when operably engaged with the cranium, provides a clearance gap between the cranium and the underside of fixation base facing the cranium.

2. The access port assembly of claim 1, configured to assist in providing a burr hole in a patient's cranium and for fixating, relative to the patient's cranium, a catheter to be deployed via the burr hole to provide fluid communication between an intracranially located cavity and an extracranially located cavity.

3. (canceled)

4. (canceled)

5. (canceled)

6. The access port assembly of claim 1, wherein the guide element is tiltably coupled with the fixation base for tilting of the guide element around at least one tilt axis relative to the fixation base.

7. The access port assembly of claim 1, wherein the guide element is tiltably coupled with the fixation base for tilting of the guide element relative to the fixation base around a plurality of different tilt axes including a first tilt axis X and a second tilt axis Y that is perpendicular to the first tilt axis X.

8. (canceled)

9. (canceled)

10. The access port assembly of claim 1, comprising a linkage clamp mechanism configured to apply pressure onto the socket of the ball-and-socket joint for frictionally securing the guide channel in a selected orientation relative to the fixation base.

11. (canceled)

12. (canceled)

13. The access port assembly of claim 1, configured for guiding a drill to create a bore hole into a patient's cranium for the deployment of a catheter through the bore hole to perform one or more of the following:
ventricular drainage; or intracranially delivering a pharmaceutical substance.

14. (canceled)

15. The access port assembly of claim 1, wherein the guide locking mechanism includes a guide clamp configured to radially apply pressure onto the external surface of a medical device received by the guide channel for frictionally securing the medical device in a selected axial position within the guide channel.

16. The access port assembly of claim 1, wherein the guide locking mechanism comprises:
a sleeve-shaped part having a longitudinal sleeve axis that coincides with the longitudinal channel guide axis, wherein the sleeve-shaped part comprises:
a base; and
a plurality of elastically clampable and circumferentially arranged arch-shaped and deflectable wings extending from the base, wherein the wings are at least partially exteriorly threaded; and
a locking ring comprising an interiorly threaded opening for cooperatively and lockingly engage with the exterior threads of the sleeve by radially applying with the wings pressure onto a medical device received in the guide channel.

17. The access port assembly of claim 16, wherein at least portion of the deflectable wings delineate an outer circumference that is larger than the inside circumference of the locking part to cause the clamping wings to be pressed inwardly by the inside thread of the locking part in direction towards the channel guide axis.

18. The access port assembly of claim 16, comprising a locking cap that includes the locking ring.

19. An access port assembly configured to assist in creating access for and accommodating a medical device extending from outside a patient body and terminating in a cavity internal to the patient body, the access port assembly comprising:
a fixation base comprising at least one fastener for securely coupling the access port assembly in a selected position to a site of interest external to the patient body;
a guide element comprising a guide channel having a longitudinal guide axis extending from an inlet to an outlet of the guide element for guiding the medical device through the guide channel,
wherein the guide element is movably coupled with the fixation base by an articulating linkage to allow selecting an orientation of the guide channel relative to the site of interest of the patient body;
an orientation locking mechanism configured to secure the guide channel in a selected orientation and to unsecure the guide channel to allow manipulation of the guide channel into a selected orientation relative to the fixation base;
a guide locking mechanism for axially securing the medical device in a desired position within the guide channel of the guide element,
wherein the articulating linkage is implemented as a ball-and-socket linkage;
wherein the guide element is movably coupled with the fixation base by an articulating linkage to allow selecting an orientation of the guide channel relative to the site of interest of the patient body; and
wherein the guide element and the articulating linkage are configured so that they are form-fittingly coupleable with each other.

20. The access port assembly of claim 19, wherein the guide element and the articulating linkage are configured as or configured to include a quick-connect mechanism allowing quick-connecting the medical device with the articulating linkage and quick-releasing the medical device from the articulating linkage.

21. A system for monitoring and controlling flow of intracranial fluid, the system comprising:
at least one first cranial inlet access port assembly,
at least one second cranial outlet access port assembly;
at least one pump device configured to impart energy onto fluid, and having a pump inlet and a pump outlet;
wherein the first inlet access port assembly and the pump outlet can be brought in fluid communication with each other by a cranial inlet catheter tubing; and
wherein the first outlet access port assembly and the pump inlet can be brought in fluid communication with each other by a cranial outlet catheter tubing;
at least one sensor configured to provide at least one sensor output relating to at least one sensed characteristic of cerebrospinal fluid pressure;
at least one processor;
at least one memory storing software code portions executable by the at least one processor to cause the system to perform the following steps:
receiving the at least one sensor output; and
controlling, based on the at least one sensor output, the operation of the at least one pump device.

22. The system of claim 21, wherein the at least one sensed characteristic includes one of the following:
fluid flow rate, fluid pressure, fluid temperature, concentration of one more target analytes in the fluid, or any combination of the aforesaid.

23. The system of claim 22, wherein the at least one liquid characteristic includes: intracranial pressure, and/or cerebrospinal fluid (CSF) pressure. |24. (Currently Amended) The system of claim 21, further comprising:
drainage tubing; and
wherein the steps further include:
controllably draining CSF to relieve elevated intracranial pressure.

25. (canceled)

26. (canceled)

27. The system of claim 21, further comprising:
a fluid reservoir that is in fluid communication with the at least one pump device for controllably providing the patient with fluid stored in the fluid reservoir via the first cranial inlet access port assembly.

28. (canceled)

29. (canceled)

30. (canceled)

31. A trajectory determination assembly configured for assisting in determining a desired trajectory of a ventricular drainage catheter through a cranial target site, the trajectory determination assembly comprising:
a first arm and a second arm that are articulated with each other to enable rotation relative to each other around a rotation axis;
wherein the first arm comprises a first light source configured to emit in a first projection orientation, which is selectable with respect to at least one first facial feature, a first projection plane to project a first line marking on the patient's head;
wherein the second arm comprises a second light source configured to emit in a second projection orientation, which is selectable with respect to at least one second facial feature, a second projection plane to project a second line marking on the patient's head; and
wherein the trajectory guide is configured such that when the first arm and the second arm are concurrently set, respectively, in the selected first and second projection orientations, the rotation axis of the first arm and the second arm coincides with the desired catheter trajectory.

32. The trajectory determination assembly of claim 31, wherein the first and the second light source are configured with respect to the first and the second arm such that an intersection between the first and the second projection plane coincides with the rotation axis.

33. (canceled)

34. (canceled)

35. (canceled)

36. (canceled)

37. (canceled)

38. A method for monitoring and controlling flow circulation of intercranial fluid through a first inlet port and inlet catheter tubing, and a second outlet port and outlet catheter tubing that are in fluid communication with the cranium of a subject, the method comprising:
receiving at least one sensor output relating to at least one sensed characteristic of cerebrospinal fluid pressure; and
controlling, based on the at least one sensor output, the operation of at least one pump device configured to impart energy onto the fluid.
